(19) United States
(12) Patent Application Publication
(10) Pub. No.: US 2015/0265250 A1
(43) Pub. Date: Sep. 24, 2015

(54) DATA RECONSTRUCTION FOR IMPROVED ULTRASOUND IMAGING

(71) Applicant: Brigham and Women's Hospital, Inc., Boston, MA (US)

(72) Inventor: Bruno Madore, Chestnut Hill, MA (US)

(21) Appl. No.: 14/373,261

(22) PCT Filed: Jan. 18, 2013
(86) PCT No.: PCT/US13/22264

§ 371 Date: Jul. 18, 2014

Related U.S. Application Data
(60) Provisional application No. 61/588,257, filed on Jan. 19, 2012.

(51) Int. Cl. A61B 8/08; A61B 8/00; A61B 5/00; G06T 11/00

(52) U.S. Cl. CPC A61B 8/5207; G06T 11/005; A61B 8/4494; A61B 8/469; A61B 5/7257; A61B 8/463; A61B 8/5269; G06T 2207/10132; G06T 2210/41

(57) ABSTRACT

A system and method for reconstructing ultrasound images provides improvements in image quality by using and digitally processing the acquired data along a plurality of dimensions. The echo signal reflected off different features in the object is reconstructed into images by solving a regularized linear system of equations that involves the geometry of the imaging transducer and of the image field-of-view. Processing can be performed ahead of time to create reconstruction matrices that can be reused indefinitely for a given transducer and field-of-view. The present invention can include a temporal encoding and decoding scheme, which includes changes in the direction of propagation and/or focusing characteris-tics of the transmitted ultrasound field from one time frame to the next, to provide improved discrimination between desired object features and artifacts.

### CROSS-REFERENCE TO RELATED APPLICATIONS

This application claims the benefit of U.S. Provisional Application Ser. No. 61/588,257, filed Jan. 19, 2012, the entire contents of the above application being incorporated herein by reference.

### BACKGROUND OF THE INVENTION

The present invention relates to the field of ultrasound imaging and, more particularly, to a system and method for improving the quality of reconstructed ultrasound images.

Ultrasound imaging is a low-cost, safe, and mobile imaging modality that is widely used in clinical radiology. Ultrasound fields can be used in various ways to produce images of objects. For example, the ultrasound transmitter may be placed on one side of the object and the ultrasound receiver on the other side, but more commonly transmitter and receiver are on the same side of the object. The transmitter and receiver typically correspond to a same piece of hardware that switches in time between a transmitter and a receiver mode of operation, and the brightness of each image pixel is a function of the amplitude, time-of-flight or frequency shift of the ultrasound reflected from the object back to the receiver.

Ultrasound transducers are devices that are meant to create a vibration, and this vibration then propagates into the imaged object in the form of an ultrasound field. The vibration typically originates from a time-varying electric field applied to a piezoelectric material, and it is transmitted to the imaged object through physical contact with the transducer. The ultrasound field can then propagate into the imaged object and interact with it. The ultrasound energy that manages to reach the receiver gives rise to electrical signals that can be converted into ultrasound images. Typically, the front of the transducer is covered with acoustic matching layers that improve the coupling between the transducer and the imaged object, to minimize reflections of the ultrasound energy as it passes from the transducer to the imaged object (during transmission) or from the imaged object back into the transducer (during signal reception). In addition, a backing block is typically located behind the piezoelectric material to reduce ringing and allow short, compact bursts of ultrasound energy to be transmitted. The signal as received by the transducer elements is often referred to as the ‘RF signal’.

When used for ultrasound imaging, a transducer typically consists of a number of elements arranged in an array and driven with different voltage waveforms. By controlling the time delay (or phase) and amplitude of the applied voltages, the ultrasound field produced by the array can be made to focus at a selected point in space, where the contributions from all elements add constructively thus maximizing the field strength at this particular location. By controlling the time delay and amplitude of the applied voltages, this focal point can be moved at different spatial locations in the imaged field-of-view. Alternately, the time delay and amplitude of the applied voltages can be adjusted so that the ultrasound field does not focus anywhere in the object, thus sonicating a large portion (or even all) of the object in a single transmit event, for fast imaging.

As indicated above, there are a number of different ways of employing an ultrasound transducer to sonicate an object and generate imaging data from it. A common strategy can be referred to as a “linear array”, whereby a small group of elements are fired in such a way as to produce an ultrasound field that travels away from the transducer, perpendicular to its surface. The system then switches to receiver mode after a short time interval. The subset of elements selected to be fired would typically form a continuous region on the transducer's surface, and this selected region gets translated across the transducer's surface during the scan to produce a corresponding series of parallel beams. Each beam is focused by adjusting the time delay (and/or phase) of the inner elements as compared to the outer elements in each subset. The time delays determine the depth of focus, and can be changed during scanning. The scan is considered complete once enough beams have been acquired to cover the desired (rectangular-shape) field-of-view. Several different transmit events are required before the field-of-view can be scanned in its entirety, as each transmitted beam typically involves one separate transmit event. Another common strategy can be referred to as a “phased array”. In this case, all of the elements of a transducer array may be used to transmit a steered ultrasound beam. A series of measurements are made at successive steering angles to scan a pie-shaped sector of the subject. The time required to conduct the entire scan is a function of the time required to make each measurement and the number of measurements required to cover the entire desired field-of-view.

Similar scanning methods may be used to acquire a three-dimensional image of the subject. The transducer in such case may be a two-dimensional array of elements which steer a beam throughout a volume of interest or linearly scan a plurality of adjacent two-dimensional slices.

At the receiver stage, when the transducer is employed to receive the ultrasound field reflected from object features, focusing can be used in a similar manner as for the transmit stage. As for the transmit stage, focusing at the receiver stage is achieved by imparting separate time delays (and/or phase shifts) and gains to the echo signal received by each transducer array element. After proper weighing and time delays are applied, the voltages produced at the transducer elements in the array are summed together such that the net signal represents the ultrasound signal reflected from a single focal point in the object. Typically, the focus point used at the receiver stage lies on the beam path that had been used at the transmission stage. The receiver is dynamically focused at a succession of ranges along the path of the transmitted beam, creating a series of points along a scan line as the reflected ultrasound waves are received. This image reconstruction process, whereby image points are obtained through a weighted sum of time-delayed signals, is often referred to as “delay-and-sum beamforming” and it can be performed very rapidly on dedicated hardware. Other operations such as time-gain-compensation (TGC), envelope detection and gridding may complete the reconstruction process. Hardware implementations of delay-and-sum beamforming have made ultrasound imaging possible at a time when computing power was insufficient for entirely-digital reconstructions to be practical. But improvements in computer technology and the introduction of scanners able to provide access to digitized RF signals have now made digital reconstructions possible. Software-based reconstructions are more flexible, and may allow some of the approximations inherited from hardware-based processing to be lifted.

In adaptive ultrasound imaging the weights in delay-and-sum beamforming are adjusted in an adaptive manner, based on the data being received, to help improve image quality and to best suit the particular object being imaged. For example, a ‘minimum variance beamforming’ method aims to find weights that minimize the L2-norm of the beamformed signal (thus making reconstructed images as dark as possible), while at the same time enforcing a constraint that signals at focus be properly reconstructed. The overall effect is to significantly suppress signals from undesired sources while preserving for the most part signals from desired sources. Such adaptive beamforming approaches are referred to here as ‘delay-weigh-and-sum beamforming’, to emphasize the fact that elaborately-selected weights are being included into the delay-and-sum beamforming process. Irrespective of the degree of sophistication involved in selecting the weights, delay-weigh-and-sum beamforming remains essentially one-dimensional in nature, as the received signal is summed over all receiver elements. Thus, further improvements are needed to further improve image processing in ultrasound operations.

### SUMMARY OF THE INVENTION

The present invention relates to systems and methods for reconstructing ultrasound images that use the whole data space acquired (all time points for all receiving elements and all transmit events for present and past time frames) when reconstructing a given image pixel, along with prior knowledge that assists in the reconstruction process. This provides improvements in image quality metrics such as spatial resolution, image contrast and artifact content.

The present invention overcomes the aforementioned drawbacks by providing a method that uses and digitally processes the acquired ultrasound data along a plurality of dimensions toward generating ultrasound images. Preferred embodiments of the invention process the data acquired by receiver elements, time points and transmit events more completely to achieve improved image quality. Data from past time frames (i.e., full images formed at prior time points) can be employed in the reconstruction process through a temporal strategy whereby the transducer-firing sequence is modified from one time frame to the next and temporal filters are applied to the reconstructed results.

In accordance with one aspect of the invention, a system and method for improving the quality of reconstructed ultrasound images is provided. The method includes acquiring ultrasound image data with a transducer array and processing this data with data processors, using a model that includes a spatially varied regularization component. Thus, instead of using conventional delay and sum beamforming, preferred embodiments of the present invention use a reconstruction matrix to compute an ultrasound image from the RF ultrasound data. In a further embodiment, a plurality of images can be created, using different sets of transmitted ultrasound fields to help label and suppress image artifacts. This can involve, for example, rotating a pulse axis and phase compensation of the detected signal. A real time filter can then be applied to remove artifacts.

Various other features of the present invention will be made apparent from the following detailed description and the drawings.

### BRIEF DESCRIPTION OF THE DRAWINGS

FIG. 1 is a block diagram of an ultrasonic imaging system which employs the present invention.

FIG. 2 is a schematic showing the dimensions involved in acquired ultrasound datasets.

FIG. 3 is a flowchart setting forth the steps for producing an improved-quality ultrasound image in accordance with the present invention.

FIGS. 4A and 4B are flowcharts detailing the processing involved toward image production.

FIGS. 5A and 5B are an acquired ultrasound dataset along with a corresponding ultrasound image.

FIGS. 6A-6C show a simulated ultrasound dataset from a point object along with a pair of ultrasound images reconstructed from these data in accordance with preferred embodiments of the invention.

FIGS. 7A-7C are a series of wavepackets as transmitted by given ultrasound transducers in given imaging geometries.

FIG. 8 is a schematic of a transducer and imaged object, to illustrate variations in signal strength.

FIGS. 9A-9E show a combination of calculated matrices and schematics illustrating how sparsity is present in the problem being solved, along with methods to use this sparsity.

FIG. 10 is a series of line plots to exemplify how sparsity and accuracy can be traded-off against each other, along with example settings.

FIG. 11 is a line plot to exemplify how regularization parameters can be optimized and set.

FIGS. 12A-12G are a series of line plots and a table assessing the ability of the present invention to improve resolution and contrast.

FIGS. 13A-13H are a series of schematics and images assessing the ability of the present invention to improve resolution and contrast.

FIG. 14 is a flowchart setting forth the steps for producing an improved-quality ultrasound image in accordance with the present invention.

FIGS. 15A-15C are a series of images assessing the ability of the present invention to reduce the artifact content and improve contrast.

FIGS. 16A-16E are a series of schematics and simulated images exemplifying various schemes wherein the transmitted ultrasound field may change from one time frame to the next as part of the present invention.

FIG. 17 is a series of images exemplifying how the present invention can be used to provide spatial localization independently of any traditional beamforming.

FIG. 18 is a schematic showing a departure from an arc shape in the received ultrasound signal, as expressed in e-t space.

FIG. 19 is a flowchart setting forth the steps for producing a map of the speed of sound.

FIG. 20 is a process sequence for processing ultrasound data in accordance with preferred embodiments of the invention.

### DESCRIPTION OF THE PREFERRED EMBODIMENT

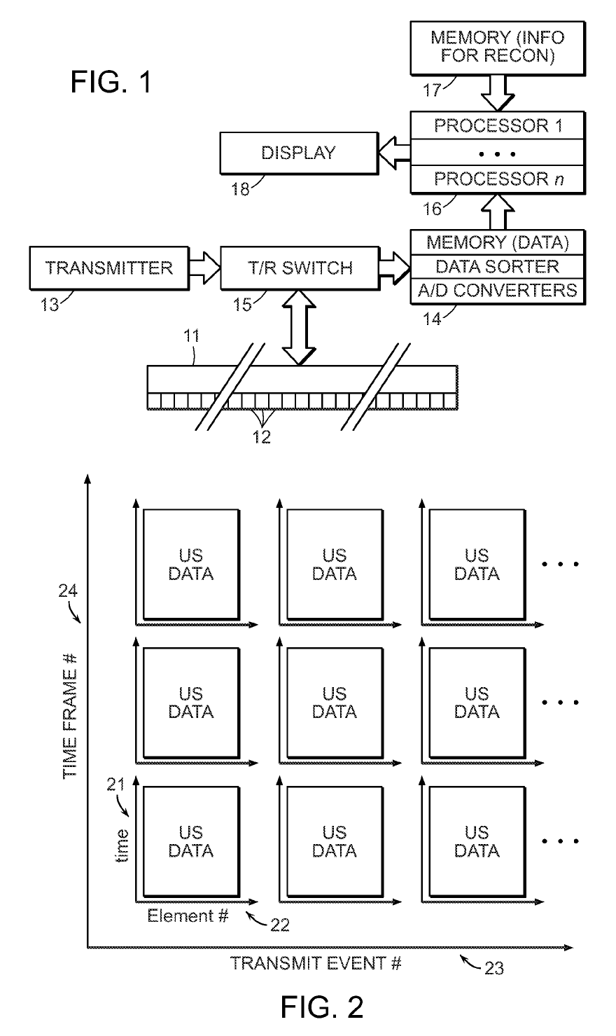

Referring particularly to FIG. 1, an ultrasound imaging system includes a transducer array 11 comprised of a plurality of separately-driven elements 12. These elements produce ultrasound energy when energized by a pulse produced by a transmitter 13. The ultrasound energy reflected back to the transducer array 11 from the subject being imaged is converted into electrical signals by the transducer elements 12. The electrical signal is sent to the receiver 14 through switches 15. A complete scan typically involves acquiring a series of echoes in which the transmitter 13 energizes the array 11 through the switches 15, which are momentarily set in a transmit mode; the switches 15 then revert to a receive mode and the received signal is sent to the receiver 14. The receiver can digitize and order the data for further processing. The received data are reconstructed into an image by the processing unit 16 and sent to a display system 18. Referring particularly to FIG. 2, the ultrasound signal gathered by the ultrasound imaging system from FIG. 1 has several dimensions: A time axis 21 captures the fact that time elapses while the reflected ultrasound waves are being received, a transducer-element axis 22 captures the fact that the transducer 11 consists of a plurality of elements 12, a transmit-event dimension 23 captures the fact that each ultrasound image can involve more than one transmit event, and a time-frame axis 24 captures the fact that a plurality of ultrasound images are typically acquired, at a frame rate of R frames per second (fps).

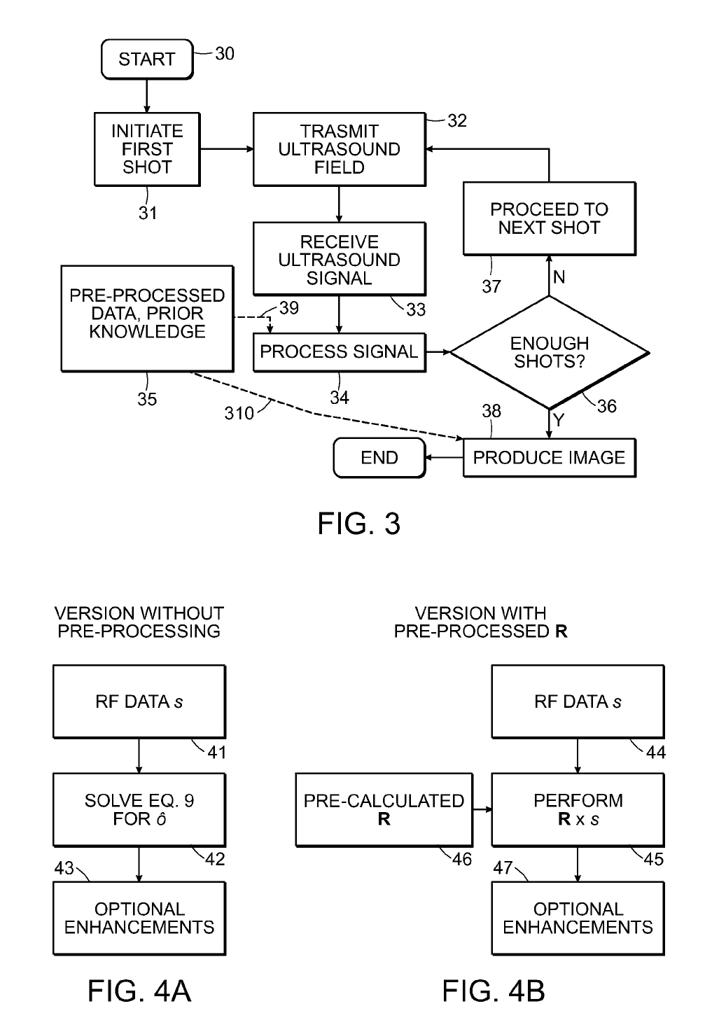

The ultrasound imaging system from FIG. 1 can be employed to perform a variety of imaging modes, including the generation of images whose overall quality has been improved in accordance with the present invention. In general, the present invention involves generating an ultrasound image from sampled and digitized data using a software-based reconstruction strategy that involves potentially all of the dimensions 21, 22, 23 and 24 from FIG. 2. Note that the process described below with reference to FIGS. 3 and 4A-4B involves the production of a single image and may be repeated as necessary to allow the production of a time series of images at video rates.

Referring to FIG. 3, the present invention begins with the generation of an ultrasound field for a first transmit event, or shot. The field transmitted in 32 can be focused at some location(s) within the imaged object (as in linear-array or phased-array imaging, for example), or not focused anywhere within the object. Ultrasound energy reflecting off features in the object and back to the ultrasound transducer generates an ultrasound signal that is received in 33 and processed in 34. Once the needed number of transmit events has been acquired, an image can be produced in 38. Typically each transmit event generates information for only part of the imaged object, and several transmit events may be needed to image the entire desired field-of-view. Ultrasound fields that are not focused anywhere in the object can, on the other hand, sonicate the entire field-of-view all at once, enabling single-shot imaging.

Typically, the processing in 34 and/or 38 relies on delay-and-sum beamforming to convert the received RF signal into image data. As described below and in FIGS. 4A-4B, an alternative reconstruction method is provided to achieve improved image quality. Parts of the processing that involve individual transmit events can be performed 34, while the final image is produced only once when all transmit events have been acquired 38. The processing may involve pre-processed information and/or prior knowledge 35, as indicated with arrows 39 and 310. Prior knowledge can take the form of a spatially varied regularization component, for example, designed to provide an appropriate amount of regularization to the signal from tissues located at different depths, as further explained herein.

The RF signal acquired in 33 can be represented in a space called here ‘e-t space’, where e′ is the receiver element dimension 22 and ‘t’ is the time dimension 21. This space can be either 2- or 3-dimensional, for 1D or 2D transducer arrays, respectively. A single e-t space matrix can be used to reconstruct a single ray, multiple rays, or even an entire image in single-shot imaging. In the notation used below and in FIGS. 4A and 4B, all of the points in a 2D (or 3D) e-t space are concatenated into a single 1D vector, s. The single-column vector s in 41 and 44 features Nₛ×Nₜ×Nₑ rows, i.e., Nₛ rows for each point in e-t space, where Nₑ is the number of points along the receiver-element dimension 22, Nₜ is the number points along the time dimension 21 and Nₛ is the number of shots used in the processing. In cases where the processing in FIGS. 4A-4B is performed once for all shots that have been acquired in 38, Nₛ may be equal to the total number of shots Nₛₕₒₜ along the transmit event dimension 23. Alternately, in cases where the processing in FIGS. 4A-4B is performed before all shots were acquired, in 34, Nₛ may be smaller than Nₛₕₒₜ. In the latter case, the result of all iterations through 34 are combined in 38 to produce the final image. The multiple shots may represent different lines in the image (1D shift from one transmit beam to the next), or different regions in the image (2D or 3D shift). In the latter case, the process of selecting focus locations for all of the Nₛₕₒₜ shots can be based, for example, on trying to maximize a function such as:

f(ρ,θ)=ΣᵢG(ρᵢ,θᵢ)×|A(ρᵢ,θᵢ)|^{a}, [1]

where A(ρᵢ,θᵢ) is the simulated amplitude of the ultrasound field for a focus location at (ρᵢ,θᵢ), i ranges from 1 to Nₛₕₒₜ, G(ρᵢ,θᵢ) is a Gaussian weighting with maximum at (ρ₁,θᵢ), and a<1.0.

Using the RF data vector s as defined above and in FIG. 4, a regular delay-and-sum beamforming reconstruction can be described as:

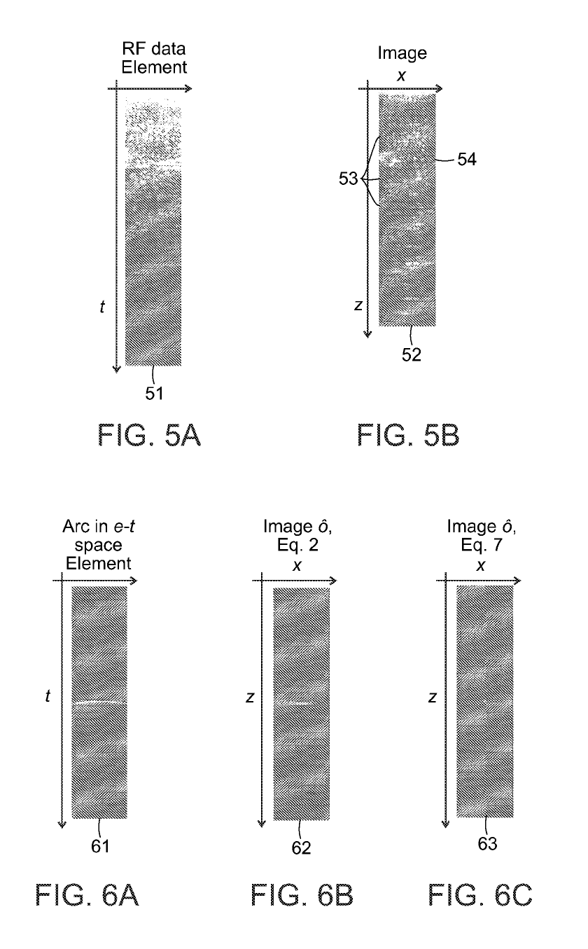

ô=A{G×V{D₀×T₀×s}}=A{G×V{R₀×s}}, [2]

where ô is the image rendering of the ‘true’ sonicated object o; it is a 1D vector, with all Nₓ×N_{z} voxels concatenated into a single column. Time gain compensation (TGC) is performed by multiplying the raw signal s with the matrix T₀, which is a diagonal square matrix featuring Nₛ×Nₜ×Nₑ rows and columns Delay-and-sum beamforming is performed by further multiplying with D₀, a matrix featuring Nₗ×Nₐₓ rows and Nₛ×Nₜ×Nₑ columns, where Nₗ is the number of lines per image and Nₐₓ is the number of points along the axial dimension. The content and nature of D₀ will be described in more details later. The operator V{•} performs envelope detection, which may involve non-linear operations and thus could not be represented in the form of a matrix multiplication. Gridding is performed through a multiplication with the matrix G featuring Nₓ×N_{z} rows and Nₗ×Nₐₓ columns. The operator A{•} represents optional data-enhancement algorithms, as described in 43 and 47. The reconstruction matrix, R₀, is given by D₀×T₀. An example of an RF dataset in e-t space and its associated reconstructed image ô (rendered in 2D) is shown in FIGS. 5A-5B. Note that the carrier frequency of the raw signal was removed in 51 for display purposes, as it would be difficult to capture using limited resolution in a small figure (the raw data was made complex and a magnitude operator was applied). The image 52 further includes graphic elements to highlight regions-of-interest (ROI) as referred to later in the text (white boxes 53 and line 54). A main goal of the present work is to improve R₀ in Eq. 2 as a means to increase the overall quality of reconstructed images ô such as that in 52.

As assumed in delay-and-sum beamforming reconstructions, the signal reflected by a single point-object takes on the shape of an arc in the corresponding e-t space RF signal 51. The location of the point-object in space determines the location and curvature of the associated arc in e-t space. For a more general object, o, the raw signal consists of a linear superposition of e-t space arcs, whereby each object point in o is associated with an e-t space arc in s. The translation of all object points into a superposition of e-t space arcs can be described as:

T₀×s=E_{arc}×o, [3]

where E_{arc} is an encoding matrix featuring Nₛ×Nₜ×Nₑ rows and Nₗ×Nₐₓ columns. The matrix E_{arc} is assembled by pasting side-by-side Nₗ×Nₐₓ column vectors that correspond to all of the different e-t space arcs associated with the Nₗ×Nₐₓ voxels to be reconstructed. The reconstruction process expressed in Eq. 2 is actually a solution to the imaging problem from Eq. 3: Multiplying both sides of Eq. 3 with E_{arc}⁺, the Moore-Penrose pseudo-inverse of E_{arc}, one obtains o≈E_{arc}⁺×T₀×s. Upon adding the envelope detection and gridding steps, one can obtain Eq. 2 from Eq. 3 given that:

D₀=E_{arc}⁺. [4]

On the other hand, the operations involved in a digital delay-and-sum beamforming reconstruction (i.e., multiplying the raw signal in e-t space with an arc, summing over all locations in e-t space, and repeating these steps for a collection of different arcs to reconstruct a collection of different image points) can be performed by multiplying the RF signal with E_{arc}^{H}, where the superscript H represents a Hermitian transpose. In other words, D₀ in Eq. 2 is given by:

D₀=E_{arc}^{H}. [5]

Combining Eqs 4 and 5 gives a relationship that captures one of the main assumptions of delay-and-sum beamforming reconstructions:

E_{arc}⁺=E_{arc}^{H}. [6]

In other words, delay-and-sum beamforming reconstructions assume that assembling all e-t space arcs together in a matrix format yields an orthogonal matrix. This assumption is very flawed, as demonstrated below.

An example is depicted in FIGS. 6A-6C where the e-t signal 61 consists of a single arc, meaning that the object o consists of a single point. However, reconstructing this arc using Eqs. 2 and 5 does not give a point-like image, but rather a broad distribution of signals 62. This is because E_{arc}^{H}, and thus D₀ through Eq. 5, is only a poor approximation to E_{arc}⁺. Instead of using the approximation from Eq. 6, the imaging problem as expressed in Eq. 3 can be better handled through a least-square solution. Equation 3 is first multiplied from the left by E_{arc}^{H} to obtain the so-called normal equations: E_{arc}^{H}×(T×s)=E_{arc}^{H}×E_{arc}×o. Inverting the square normal matrix E_{arc}^{H}×E_{arc} and multiplying from the left with (E_{arc}^{H}×E_{arc})⁻¹ allows o to be estimated: ô=(E_{arc}^{H}×E_{arc})⁻¹×E_{arc}^{H}×(T×s). Upon the addition of a damped least-squares regularization term λ²L, and the insertion of Ψ⁻¹ as part of a preconditioning term E_{arc}^{H}λΨ⁻¹, an equation is obtained which exhibits the well-known form of a least-square solution:

D₁=(E_{arc}^{H}×Ψ⁻×E_{arc}+λ²L)⁻¹×E_{arc}^{H}×Ψ⁻¹,

ô=G×V{D₁×T₁×s}=G×V{R₁×s}. [7]

The signal ‘s’ may here include both legitimate and noise-related components, and ô is a least-square estimate of the actual object ‘o’. The setting of both the pre-conditioning term E_{arc}^{H}×λ⁻¹ and of the regularization term λ²L involves prior knowledge, as indicated in 35. The image 63 was reconstructed using Eq. 7. Compared to image 62, image 63 presents a much more compact signal distribution and a greatly improved rendering of the point-object. But even though images reconstructed using the R₁ matrix (e.g., image 63) may prove greatly superior to those reconstructed with delay-and-sum beamforming and the associated R₀ matrix (e.g., image 62) when dealing with artificial e-t space data such as those in 61, such improvements are typically not duplicated when using more realistic data. The reason for this discrepancy is explored in more detail below.

Datasets acquired from a single point-like object do not actually look like a simple arc in e-t space. In an actual dataset, the arc 61 can be convolved with a wavepacket along the time dimension 21, whereby the shape of the wavepacket depends mostly on the voltage waveform used at the transmit stage and on the frequency response of the transducer elements. A wavepacket has both positive and negative lobes, while the arc 61 was entirely positive. Even though the delay-and-sum beamforming assumption in Eq. 6 is very inaccurate, negative errors stemming from negative lobes largely cancel positive errors from positive lobes. For this reason, delay-and-sum beamforming tends to work reasonably well for real-life signals, even though it may mostly fail for artificial data such as those in 61. Nevertheless there is room for improvement, but the scale of the improvement cannot be expected to prove as dramatic as a comparison of images 62 and 63 might suggest.

The reconstruction process from Eq. 7 avoids the approximation made by delay-and-sum beamforming as expressed in Eq. 6, but it remains inadequate because it is based on E_{arc}, and thus assumes that object points to give rise to arcs in e-t space. While E_{arc} associates each point in the object o with an arc in the raw signal s through Eq. 3, an alternate encoding matrix E_{wav} associates each point with a wavepacket function instead. Because E_{wav} features several non-zero time points per receiver element, the reconstruction process truly becomes two- or even three-dimensional in nature, as whole areas of e-t space with dimensions 21 and 22 may get involved for potentially all transmit events along dimension 23 in the reconstruction of any given pixel location, as opposed to one-dimensional arc-shaped curves as in delay-and-sum beamforming. The solution presented in Eq. 7 is duplicated in Eq. 8 below, but it now involves a more accurate model relying on E_{wav} rather than E_{arc}:

D₂=(E_{wav}^{H}×Ψ⁻¹×E_{wav}+λ²L)⁻¹×E_{wav}^{H}×Ψ⁻¹,

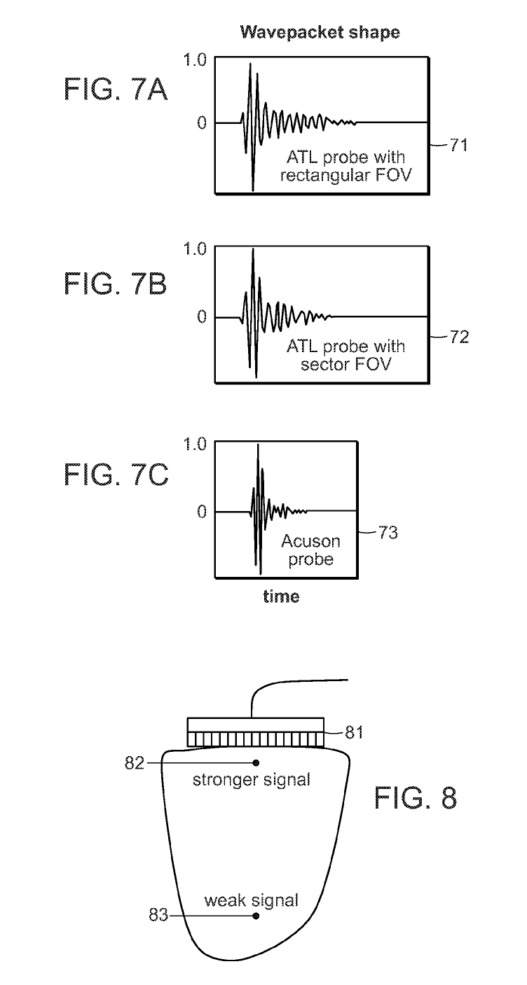

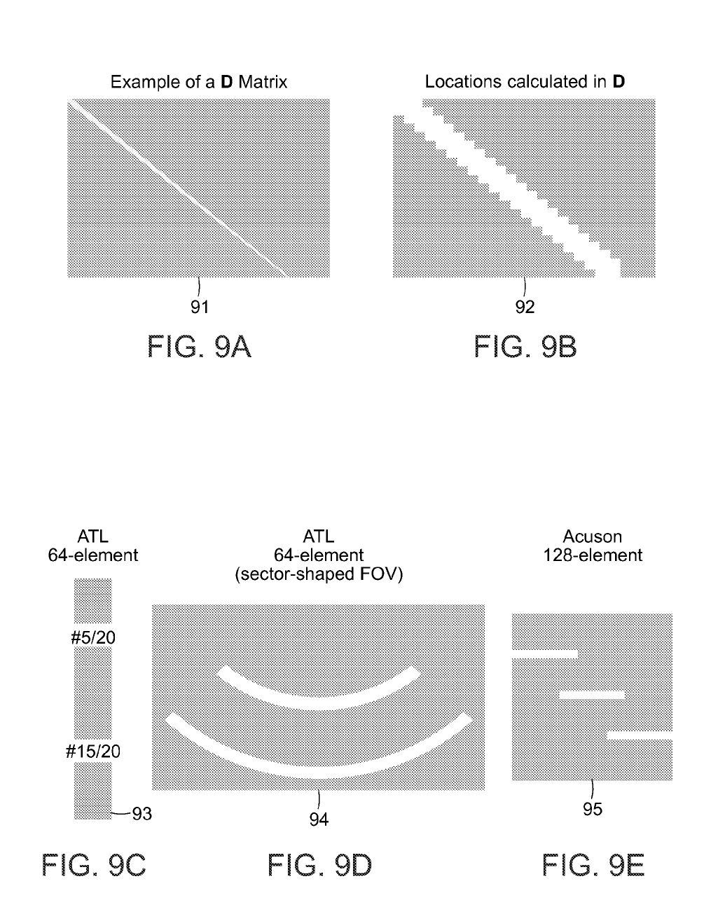

ô=D₂×T₂×s=R₂×s, [8]

where the TGC term T₂ may be equated to T₁ in Eq. 7, T₂=T₁. The main difference between E_{wav} and E_{arc} is that unlike the latter, the former includes prior information about the wavepacket or pulse transmitted by the transducer, as exemplified in FIGS. 7A-7C for three different combinations of transducer and field-of-view geometries. The transmitted wavepacket 71, 72 and 73 was obtained from a single transducer element, during a one-time reference scan, using a phantom consisting of a metal wire in a water tank.

Note that no envelope detection and no gridding operation are required in Eq. 8, unlike in Eqs. 2 and 7. As E_{wav} already contains information about the shape of the wavepacket, envelope detection is effectively performed when multiplying by D₂. Furthermore, because a separate envelope detection step is not required, there is no reason anymore for reconstructing image voxels along ray beams. Accordingly, the Nᵥₒₓ reconstructed voxels may lie directly onto a Cartesian grid, removing the need for a separate gridding step. For a rectangular FOV, the number of reconstructed voxels Nᵥₒₓ is simply equal to Nₓ×N_{z}, while for a sector-shaped FOV, it is only about half as much (because of the near-triangular shape of the FOV). As shown in detail herein, a prior measurement of the wavepacket shape, for a given combination of voltage waveform and transducer array, can be used toward generating E_{wav}. Note that unlike E_{arc}, E_{wav} is complex.

The regularization term λ²L in Eq. 8 controls the trade-off between data consistency and error amplification. Whenever the available data can be considered reliable and that its signal-to-noise (SNR) ratio is high, less regularization may be needed. On the other hand, when data are less reliable and SNR is lower, a greater amount of regularization is needed to prevent errors and noise getting amplified in the reconstruction process, negatively impacting image quality. FIG. 8 emphasizes the fact that in ultrasound imaging, the ultrasound field tends to decay very rapidly as it travels in the object. Signals from a reflector at a shallow location 82 are expected to be much stronger than that of a similar reflector at a deeper location 83, because the ultrasound field quickly decays as it travels to and from the more distant location 83. Given that a signal may differ in intensity by orders of magnitude depending on where it reflected from, not all signals can be expected to feature similar SNR and overall reliability. For this reason, not all of the received signals need the same amount of regularization. As a general rule, signals from deeper locations within the object and found at larger t values along the time axis 21 need more regularization than signals at smaller t values. Adjusting the regularization term λ²L so that it provides the right amount of regularization to all received signals can be a crucial step toward reconstructing images of good quality. To do so, λ²L must be a spatially varied regularization component, meaning that L is not an identity matrix I. For example, L may be a diagonal matrix with entries that are not constant (unequal elements) along the diagonal. More details on ways to adjust the λ²L terms are described herein in further detail.

Equation 9 below is the final step of the present process. The index ‘2’ from Eq. 8 can be dropped without ambiguity, and a scaling term (I+λ²L) is introduced to compensate for scaling effects from the λ²L regularization term:

D=(E_{wav}^{H}×Ψ⁻¹×E_{wav}+λ²L)⁻¹×(I+λ²L)×E_{wav}^{H}×Ψ⁻¹,

ô=D×T×s=R×s. [9]

As shown in 42, Eq. 9 can be solved numerically for a given s in 41. Alternately, the matrix R can be calculated beforehand as in 46 through an explicit inversion of the term (E_{wav}^{H}×Ψ⁻¹×E_{wav}+λ²L), so that the image data 6 can be generated in 45 by simply multiplying R with s. While explicitly calculating R may be a very computer-intensive operation, it can be done once-and-for-all for a given field of view setting, transmitter 13 waveform and transducer geometry. Thus, given an expressly defined set of pulse parameters for each of a plurality of different transducer arrays, a reconstruction matrix is formed and stored. In comparison, numerically solving Eq. 9 for a given s can be much faster, but such solution is preferably repeated for a plurality of new incoming RF signals s corresponding to different time frames 24 and optionally using different transmit-events 23 as well. The amount of computing power available can determine between the two versions in FIGS. 4A and 4B, as the operation 45 is much less computer intensive and can be executed much faster than operation 42.

In cases where the explicit calculation of R in 46 is performed, there are strategies to help reduce computing time and memory requirements. The matrices involved in Eq. 9 tend to be very sparse, see for example the matrix D in 91, and accordingly sparsity can be exploited and maintained throughout the processing leading to ô. Strategies to ensure sparsity include: A) As shown in FIGS. 7A-7C using black curves, the wavepackets employed as prior knowledge when constructing E_{wav} can be truncated in time so as to keep only N_{wpts} non-zero points, to help keep E_{wav} sparse. B) Instead of solving for all of D in one pass, the areas of D where non-zero values are expected (see 91) can be covered using a series of N_{patch} overlapping regions, each one only a small fraction of D in size (see 92). Results from different patches in the overlapping regions get simply combined through a weighted sum. In the image plane, these patches or data graphs can be thought of as groups of voxels that are located roughly a same distance away from the (virtual in this example) transmit focus. For rectangular-FOV geometries, different patches simply correspond to different z locations (see 93) and additional reductions in processing requirements can be achieved by further sub-dividing the x-axis as well (see 95), while for sector-shaped FOV geometries they correspond to arc-shaped regions in the x-z plane (see 94). Alternatively, these patches can be understood as square sub-regions along the diagonal of the square matrix (E_{wav}^{H}×E_{wav}+λ²L)⁻¹, which are mapped onto the non-square D and R matrices through the multiplication with E_{wav}^{H} in Eq. 9C) Once all patches or data groups are assembled into a D or an R matrix, the result gets thresholded so that only the largest N_{nz} values may remain non-zero. Preliminary thresholding on individual patches may also be performed. Smaller settings for N_{nz} lead to sparser R matrices and shorter reconstruction times in 45, but potentially less accurate image results. The need for fast reconstructions must be weighed against the need for image accuracy.

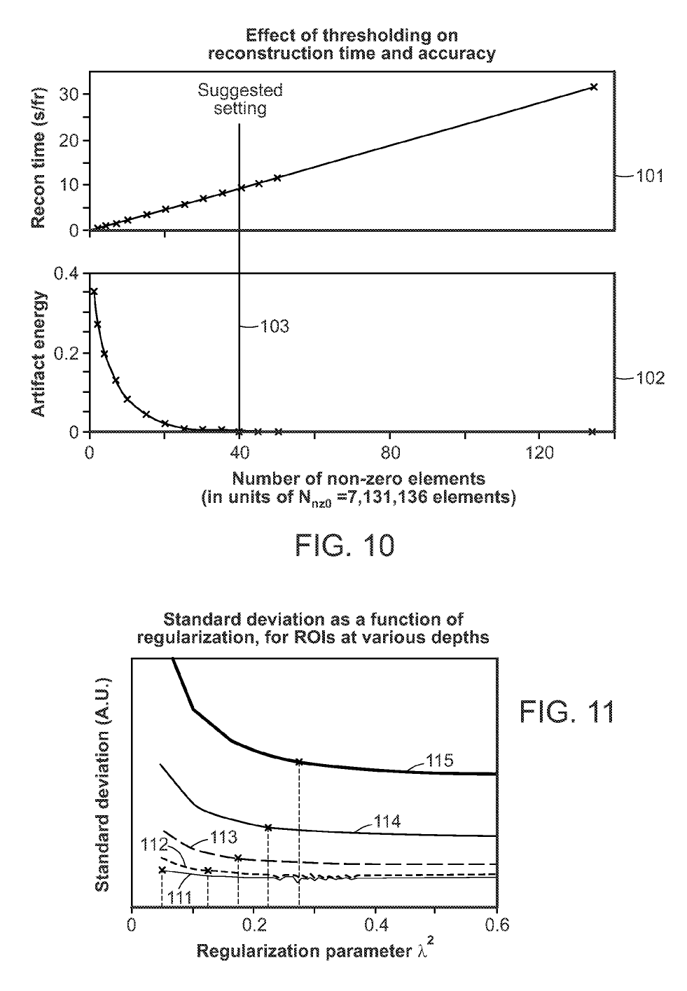

An example of the tradeoff between reconstruction speed and accuracy is shown in FIG. 10. Dataset 51 was reconstructed using a number of different values for N_{nz}, with N_{patch}=20. Reconstruction time is shown in 101, and the artifact energy as defined by

$${E}_{Nnz} = ( {\sum }_{voxels} {\vert {\hat{o}}_{Nnz} - {\hat{o}}_{ref} \vert}^{2} ) \text{/} ( {\sum }_{voxels} {\vert {\hat{o}}_{ref} \vert}^{2} ) ,$$

where ô_{Nnz} and ô_{ref} were obtained with and without thresholding, respectively, is shown in 102. The horizontal axis in FIG. 10 is expressed in terms of N_{nz0}=7131136, the number of non-zero elements in R₀, as obtained when performing a regular delay-and-sum beamforming reconstruction on the same data (Eq. 2). The number of non-zero elements in R, N_{nz}, should be made as small as possible to achieve shorter reconstruction times, but kept large enough to avoid significant penalties in terms of image quality and artifact energy (see suggested setting 103).

FIG. 11 relates to adjusting the spatially varied regularization component λ²L, as found in Eq. 9. As part of the adjusting process, dataset 51 was reconstructed a plurality of times while varying the value of the scalar λ² and using L=I, an identity matrix. Although a single time frame was shown in FIG. 1, the full dataset actually featured 50 time frames and a standard deviation along the time-frame axis was calculated. The standard deviation was spatially averaged over five regions of interest 53, and is shown in FIG. 11 as a function of λ². For each curve 111 through 115 in FIG. 11, an ‘×’ indicates the amount of regularization that appears to be roughly the smallest λ² values that can be used, while still avoiding significant noise increases. As a general rule, λ² can be kept as small as possible to avoid blurring, but large enough to avoid noise amplification if/when the system becomes ill conditioned. Notice from FIG. 11 that curves 111 to 115, which correspond to ROIs located at different depths in the object, need clearly-different levels of regularization. A depth-dependent regularization term λ²L is sought, with L≠I, whereby an appropriate amount of regularization is provided at all depths. The term λ²L was set so as to provide a depth-dependent regularization as indicated by the ‘X’ symbols seen on curves 111 through 115.

For example, defining a normalized depth r=(\sqrt{x²+(z+d_{vf})²}−d_{vf})/w_{probe}, where d_{vf} is the distance to the virtual focus behind the transducer and w_{probe} is the width of the transducer probe in the x direction, the location of the ‘×’ marks in FIG. 5 correspond to λ²=r/20. Because having no regularization at r=0 might be problematic, a minimum value of 0.1 can be used for λ², so that λ²=max(r/20, 0.1). In practice, λ² was equated to the constant part of this expression, λ²=1/20, while the diagonal of the Nᵥₒₓ by Nᵥₒₓ matrix L was equated to the variable part, so that λ²×diag(L)=max(rⱼ/20, 0.1), where j ranges from 1 to Nᵥₒₓ. More generally, this expression cannot be expected to hold for all FOV and probe geometries, and when an ATL P4-2 probe in a sector-FOV mode rather than the rectangular-FOV mode employed in FIG. 5, a much larger number of voxels get reconstructed from essentially the same number of raw-data points, suggesting that conditioning might be degraded and that a higher level of regularization might prove appropriate. For both sector-FOV results with our ATL probe and rectangular-FOV results with our Acuson probe, regularization was scaled up by a factor of 4 compared to the expression above, leading to λ²×diag(L)=max(rⱼ/5, 0.1).

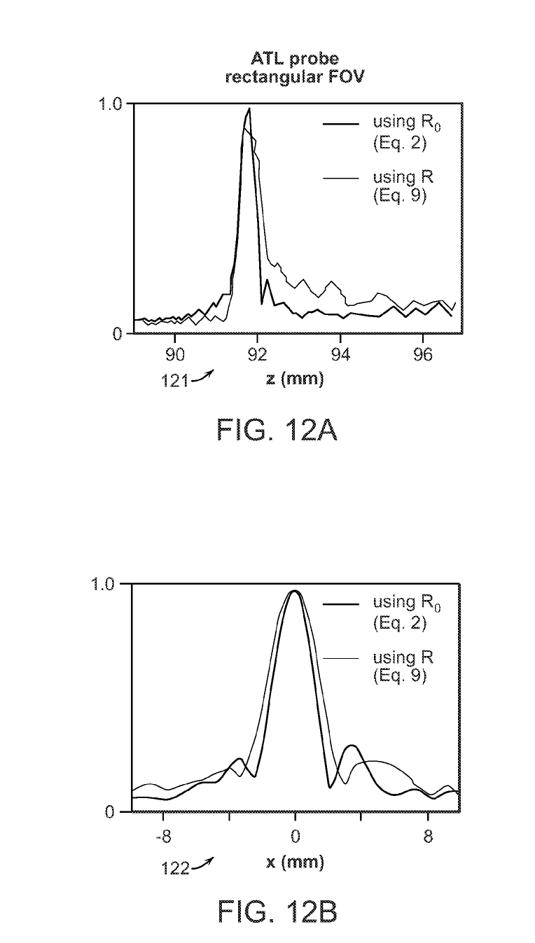

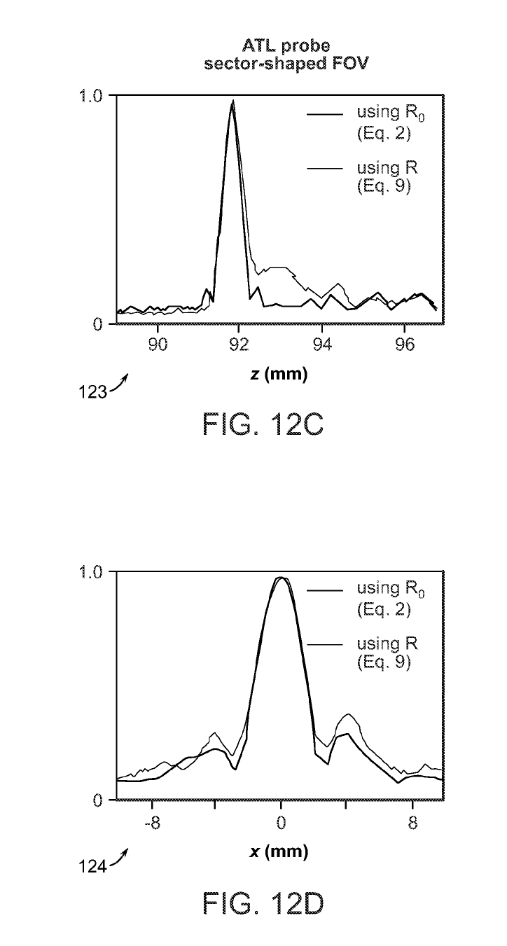

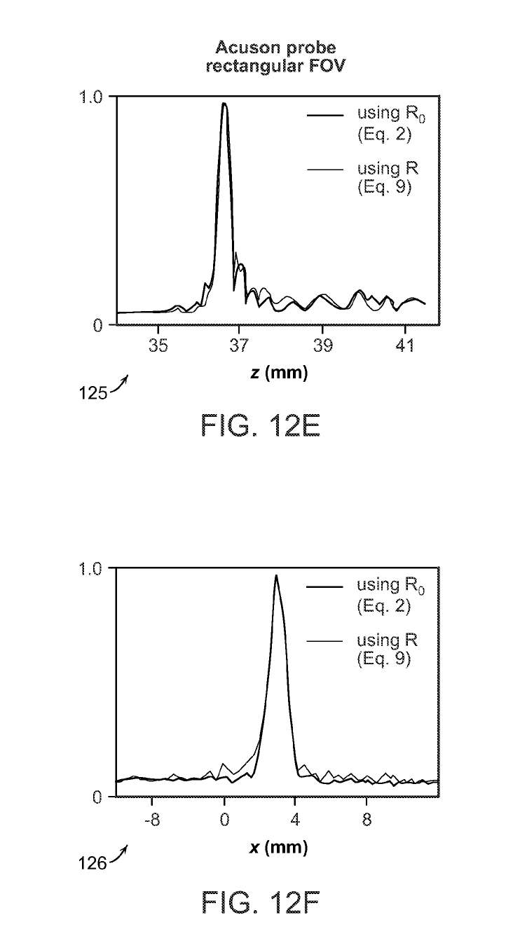

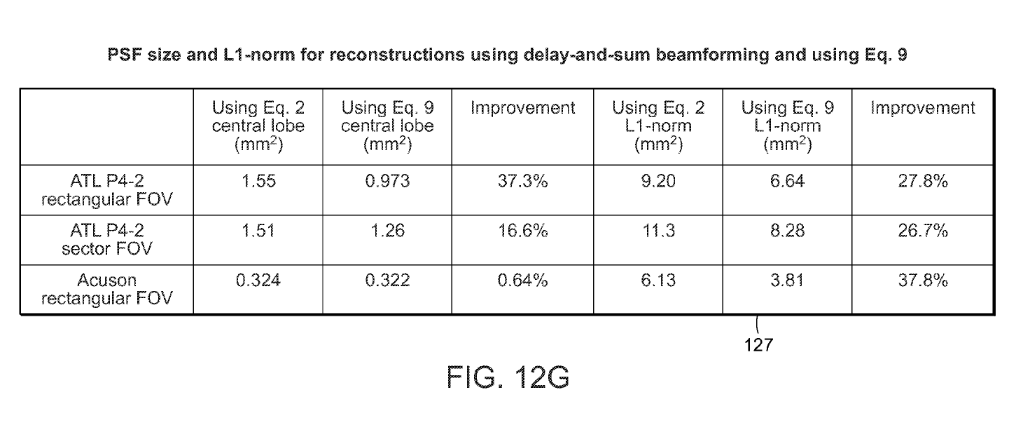

FIGS. 12A-12G show results obtained from a metal-wire phantom using single-shot imaging. The imaging was performed with our ATL probe both with a rectangular-shaped and with a sector-shaped FOV, and our Acuson probe with a rectangular-shaped FOV. The acquired datasets were reconstructed using both delay-and-sum beamforming (Eqs 2 and 5) and using Eq. 9. Because the water-wire transition had small spatial extent, resulting images are interpreted as a point-spread-function (PSF). The full-width-half-max (FWHM) of the signal distribution was measured along the x and z axes, giving FWHMₓ and FWHM_{z}. The size of the PSF was interpreted here as the size of its central lobe, as approximated by (π×FWHMₓ×FWHM_{z}/4). A second measurement was performed which involved the whole PSF distribution, rather than only its central lobe: After normalizing the peak signal at the wire's location to 1.0 and multiplying with the voxel area, the absolute value of the PSF signal was summed over an ROI about 3 cm-wide and centered at the wire. The result can be understood as the minimum area, in mm², that is required to store all PSF signal without exceeding the original peak value anywhere. This measure corresponds to the L1-norm of the PSF, and along with the size of the central lobe it was used here to compare PSF results obtained from different reconstruction methods. Profiles of the PSFs along the x and z directions can be seen in 121 through 126, and reconstructions from delay-and-sum beamforming (gray lines) can be compared to results from Eq. 9 (black lines). Improvements in terms of PSF size and L1-norm are summarized in Table 127. The size of the PSF was reduced by up to 37% (ATL probe with rectangular FOV), while the L1-norm of the PSF was reduced by up to 38% (Acuson probe with rectangular FOV).

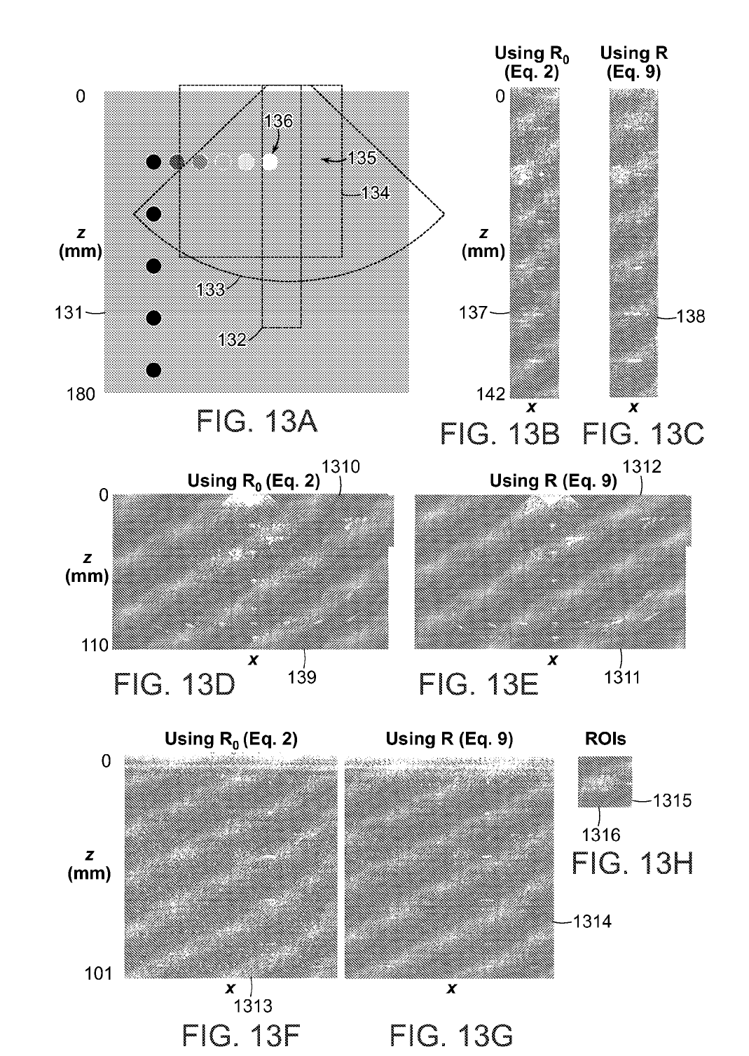

FIGS. 13A-13H show images obtained using a phantom 131 manufactured by CIRS (Norfolk, Va., USA) model 054GS, using the same probes and FOV geometry as for the metal-wire phantom from FIG. 12. Image 138 acquired with FOV 132 appears to feature greater spatial resolution than its delay-and-sum beamforming counterpart 137. Similarly, spatial resolution appears superior in image 1311 acquired with FOV 133 than in its counterpart image 139. This statement appears to be confirmed when comparing more specifically the zoomed regions 1312 and 1310 of the resolution targets 135. With S_{C} the mean signal over the circular ROI 1315 and S_{R} the mean signal over the ring-shaped ROI 1316, contrast for the hyperechoic circular region 136 was defined as (S_{C}−S_{R})/(S_{C}+S_{R}). Because a lesser amount of signal was allowed to ‘bleed’ away from hyperechoic region 136 when using Eq. 9 for the reconstruction, contrast as defined above was increased by 29.2% in 1314 compared to the delay-and-sum beamforming results 1313, from a value of 0.248 to a value of 0.320. Note that the amounts of spatial resolution and contrast improvements reported above do not represent a theoretical limit for the present method but merely what can be achieved with the present implementation. In principle at least, in a noiseless case where the encoding matrix is known, the PSF can be reduced to little more than a delta function. In more realistic situations, limitations on the achievable spatial resolution result from inaccuracies in the encoding matrix, the effect of regularization, and limits on both memory usage and reconstruction time.

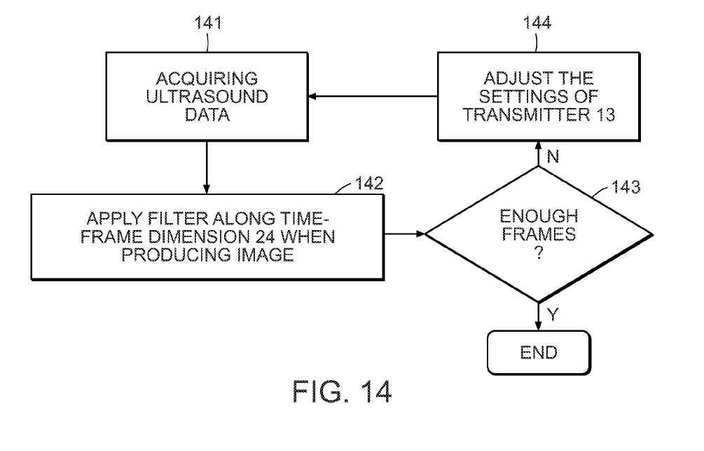

The processing in FIG. 3 involved a plurality of time points 21, a plurality of transducer elements 22 and potentially a plurality of transmit events 23. As described in FIG. 14, the production of an image can also involve a plurality of time frames 24. While ultrasound measurements typically involve acquiring a large number of time frames at a frame rate of about 30 fps or so, all time frames are typically acquired one after the other in essentially the same manner. In contrast, step 144 ensures that all time frames are not acquired in the same manner, so that artifacts may be time-labeled, identified and suppressed. The method from FIG. 14, when combined with that of FIGS. 3 and 4A-4B, allows information from anywhere along dimensions 21, 22, 23 and 24 in the acquired data to be exploited, in principle at least, toward reconstructing any given image pixel. Image quality improvements are obtained as correlations between neighboring pixels and frames are accounted for and resolved. This more flexible approach may conceivably lend itself to the modeling and correction of various effects and artifacts. Possible examples could conceivably include multiple reflections, and the effect of proximal voxels (i.e., close to the ultrasound transducer) on more distant voxels.

The use of information from a plurality of time frames, as described in FIG. 14, primarily aims to remove signals that failed the spatial-encoding process and negatively impact image contrast. In a single-shot acquisition, the modifications 144 to the transmitter may involve rotating the axis of propagation of the ultrasound field by φ(τ), where τ is the time-frame number 24. The time taken by the propagating field to reach any (x,z) location is a function of φ, and for this reason the phase of the reconstructed object varies as φ is varied. More specifically, a phase term Φ(x,z,τ)∝φ(τ)·tan⁻¹(x/(z+dvf)) is imposed onto the object, where dvf is the distance to the virtual focus (behind the tranducer). A reconstructed image I(x,z,τ) gets phase-corrected through:

I_{pc}(x,z,τ)=I(x,z,τ)×exp(−iΦ(x,z,τ)). (10)

Once the phase correction has been applied, all legitimate object signals are free of any φ-related phase variations. Artifact-related signals, on the other hand, may still undergo φ-related phase changes.

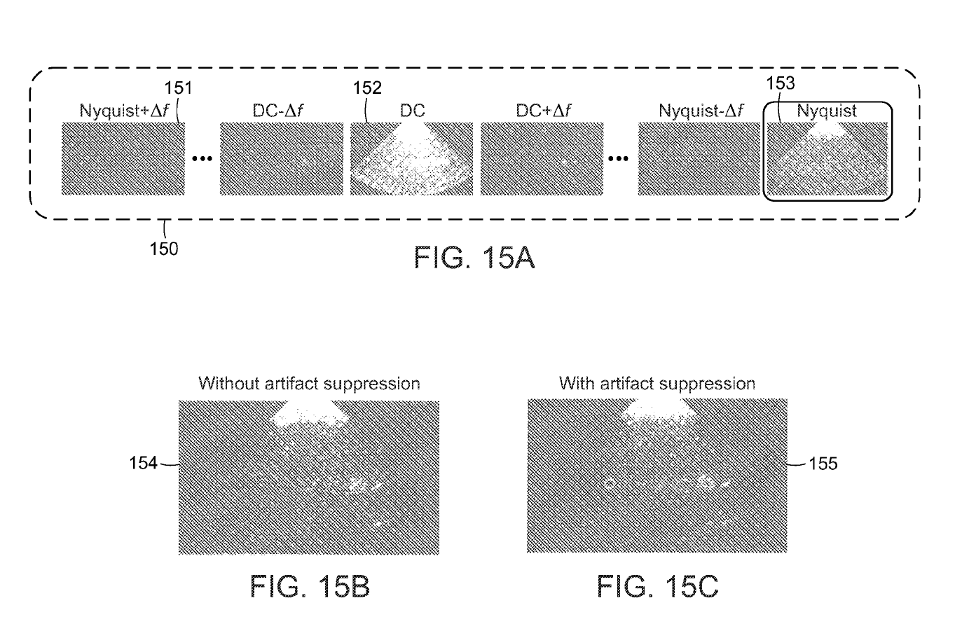

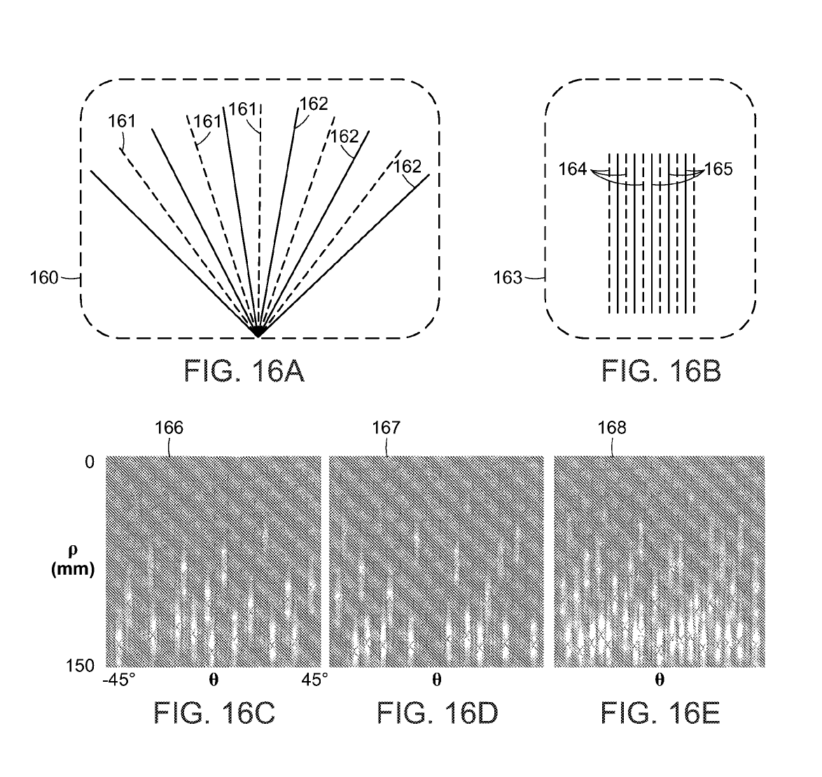

In FIGS. 15A-15C, the axis of propagation of the ultrasound field was rotated by φ(τ) with respect to the x=0 axis from one time frame to the next, and a scheme defined by φ(1)=1°, φ(2)=−1°, φ(3)=1°, φ(4)=−1°, with a periodicity of 2 frames, was employed. The images were reconstructed using Eq. 9 (two different R matrices were used, one for frames with φ(τ)=1° and another for frames with φ(τ)=−1°), although delay-and-sum beamforming could have been used instead if so desired. The resulting images were phase-corrected through Eq. 10, and Fourier transformed along the time frame 24 dimension. Six of the resulting temporal frequencies are shown in 150, including the DC 152 and Nyquist 153 frequencies, and the same windowing level was used for all of them. Because discrete Fourier transforms are periodic, the (Nyquist+Δf) frequency point 151 is in fact a neighbor of the Nyquist frequency point 153. The most important features that can be noted from FIGS. 15A-15C are that: 1) Unlike all other non-DC frequencies, the Nyquist frequency 153 contains considerable amounts of signal, and 2) the signal at Nyquist 153 does not bear any clear resemblance with the actual object as shown in 154. In other words, the acquisition scheme involving φ(τ) rotations combined with the phase correction from Eq. 10 successfully achieved a partial separation of artifacts (at Nyquist 153) and object-related signals, enabling some degree of artifact suppression.

Because φ(τ) has a 2-frame periodicity in the example from FIGS. 15A-15C, time-labeled artifacts are expected to have their value flip-flop from frame to frame between two different values. A revised version of the image 154 is obtained in 155 by applying a real-time filter F_{Ny}{•} to the data, which features a narrow stopband region centered at the Nyquist frequency 153:

I_{clean}(x,z,τ)=F_{Ny}{B{I_{pc}(x,z,τ)}}. (11)

An optional operator B{•} was included in Eq. 11, which can for example, consist of a magnitude operator if only magnitude corrections were desired. In such case, the phase correction from Eq. 10 is unnecessary. In FIGS. 15A-15C no operator B{•} was employed (i.e., identity operator).

The filter F_{Ny}{•} was applied to remove the Nyquist frequency 153 and an inverse Fourier transform was then applied to bring the signal back to the temporal domain. The first time frame in the (artifact-suppressed) series of images is shown in 155. The effectiveness of the method can be tested by comparing the signal level in hypoechoic region 156 with and without artifact suppression. As a result of applying F_{Ny}{•}, images are obtained that feature substantially reduced artifact levels. Signal in the hypoechoic region 156 was reduced by 34% in image 155 as compared to image 154, even though only a fourtieth of the temporal frequency bandwidth was filtered out (i.e., very minor reduction in temporal resolution by only 2.5%). This improvement was achieved by removing artifacts that had been time-labeled to the Nyquist frequency 153. While rotations in the transmitted field have been used in the past to help improve image quality through coherent compounding of image data, reductions in temporal resolution by 50% or more are then needed to achieve similar image-quality benefits as obtained in FIGS. 15A-15C. In contrast, a reduction in temporal resolution by only 2.5% was needed using the approach as described above. The narrowness of the filter used here, characterized by a full-width-half-maximum (FWHM) much below 50% of the sampled bandwidth, is a main characteristic of the proposed approach and the chief reason why it has such a small effect on temporal resolution.

In the implementation as presented in FIG. 15, only the highest (Nyquist) frequency region 153 was effectively used as a trash bin toward storing time-labeled artifacts. This is because the setting 144 of the transmitter repeated itself every two time frames along the time frame axis 24. For example, using a φ(τ) with a 3-frame periodicity instead, both the (2/3)×Nyquist and (−2/3)×Nyquist regions can be employed to this end, and a more general filter F{•} can be required in place of F_{Ny}{•}. More generally, with a periodicity of Nₚₑᵣ frames, the time-labeled artifacts appear at Nₚₑᵣ equidistant locations within the sampled bandwidth, one of these locations being the DC frequency 152. Because the DC frequency 152 typically features high levels of desired signals, the small amount of time-labeled artifacts present at DC is typically left alone, as the risk of negatively impacting the desired signal there is too great. On the other hand, at the (Nₚₑᵣ−1) other (non-DC) frequency locations where time-labeled artifacts are expected, desired signals may fall below a threshold whereby suppressing the artifacts becomes a worthwhile option. In FIG. 15, with Nₚₑᵣ=2, suppressing the Nyquist frequency 153 proved beneficial because relatively large amounts of artifacts and relatively low amounts of desired signals were found there. With BW the sampled bandwidth and j an integer, narrow filters to suppress signals at frequencies j×BW/Nₚₑᵣ can be applied whenever the expected signal intensity falls below a certain threshold, thus reducing the artifact content at little cost in temporal resolution. Keeping the filters very narrow, with a FWHM much below 50% of the sampled bandwidth, is crucial to ensure that the temporal resolution and frame rate is not substantially impacted. Non-periodic variations in the setting 144 of the transducer can also be imagined, leading to more complicated filter designs that may not have a compact shape in the frequency domain. The filter as described here is meant to potentially include F{•}, B{•}, as well as the phase correction from Eq. 10.

In the case of multi-shot imaging, the modifications 144 to the transmitter may involve choosing different sets of focus locations for the Nₛₕₒₜ transmit events 23. In a phased-array imaging case 160, the focus locations can be changed from one time frame to the next, so that even time frames involve beams 161, while odd time frames involve the interleaved beams 162, for example. Similarly, in linear array imaging, the focus locations can be changed from one time frame to the next so that even time frames involve beams 164 while odd time frames involve the interleaved beams 165, for example. Alternatively, multi-shot imaging can involve distributing focal regions all over the imaged FOV, rather than at a constant depth. The proposed temporal method involves alternating between different sets of focus locations, which can be selected through Eq. 1. An example is provided in 166 for even time frames and 167 for odd time frames, where the actual focus locations are marked by ‘X’ symbols while f(ρ,θ) from Eq. 1 is shown in grayscale in the background. Using the method from FIG. 14, image quality comparable to the one that can be obtained with sampling scheme 168 is obtained, even though 168 requires twice as many shots as 166 or 167. The focus locations in 166 through 168 were found by first optimizing f(ρ,θ) from Eq. 1 using a=½ for the full set 168, which was then split into two groups 166 and 167 so that f values for both subgroups is as similar and large as possible. As in the single-shot case above, cases where Nₚₑᵣ>2 can be employed, as well as cases where variations in the setting 144 of the transmitter can also be non-periodic. The filter is then varied accordingly, as the setting 144 and the filter design employed are linked and must be kept consistent with each other.

Note that the method in FIG. 14 can be used to achieve spatial localization even in the absence of any beamforming. The phantom from FIGS. 13A-13H was scanned with the technique from FIG. 14, and the phase correction for one specific location (xₒ,zₒ) was applied to all locations, such that Φₒ(τ)=(xₒ,zₒ,τ). Eq. 10 is then replaced with:

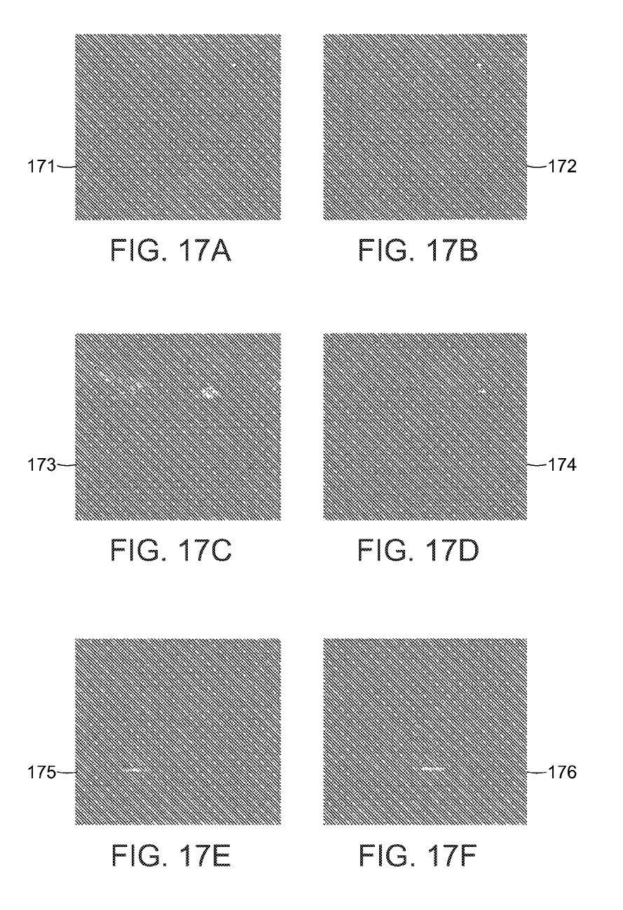

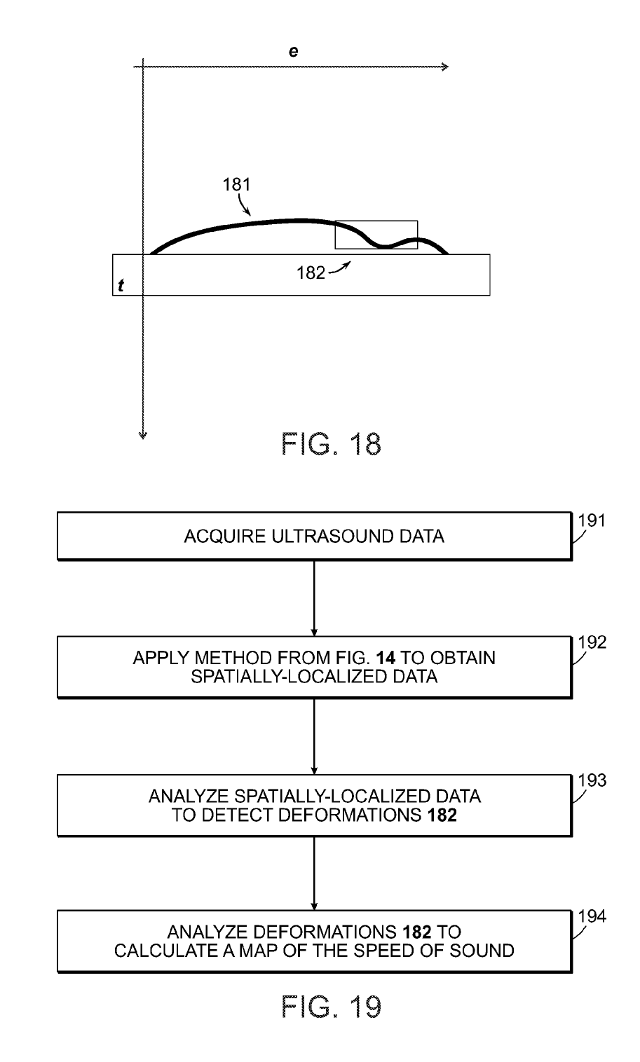

Kₒ(e,t,τ)=K(e,t,τ)×exp(−iΦₒ(τ)). (12)

where K(e,t,τ) represents the acquired e-t space data. Filtering along t was also included to help define depth. After applying a low-pass filter F{•} centered at DC, data are obtained in e-t space that feature some degree of spatial localization, as most of the signal pertains to the general area around the (xₒ,zₒ) location. This can be verified by applying delay-and-sum beamforming to reconstruct the e-t data into an image. Examples of such images are shown in FIG. 17, images 171 through 176. Note that each image shows signal mostly from one fairly-well defined region of the whole FOV. In other words, some degree of spatial localization can be obtained using the algorithm in FIG. 14, in a manner that does not depend on any traditional form of beamforming. This approach is useful when trying to detect and correct for problems that affect the accuracy of beamforming, as is the case in the presence of spatial variations in the speed of sound.

As depicted in FIG. 18, variations in the speed of sound can cause the received signal, as depicted in e-t space, to have features 182 that depart from the expected arc shape. As described in FIG. 19, data can be analyzed to detect such deformations, and these deformations can be analyzed to reveal how the speed of sound varies spatially. A spatial map of the speed of sound can be generated given that deformations such as 182 can be appropriately detected and quantified.

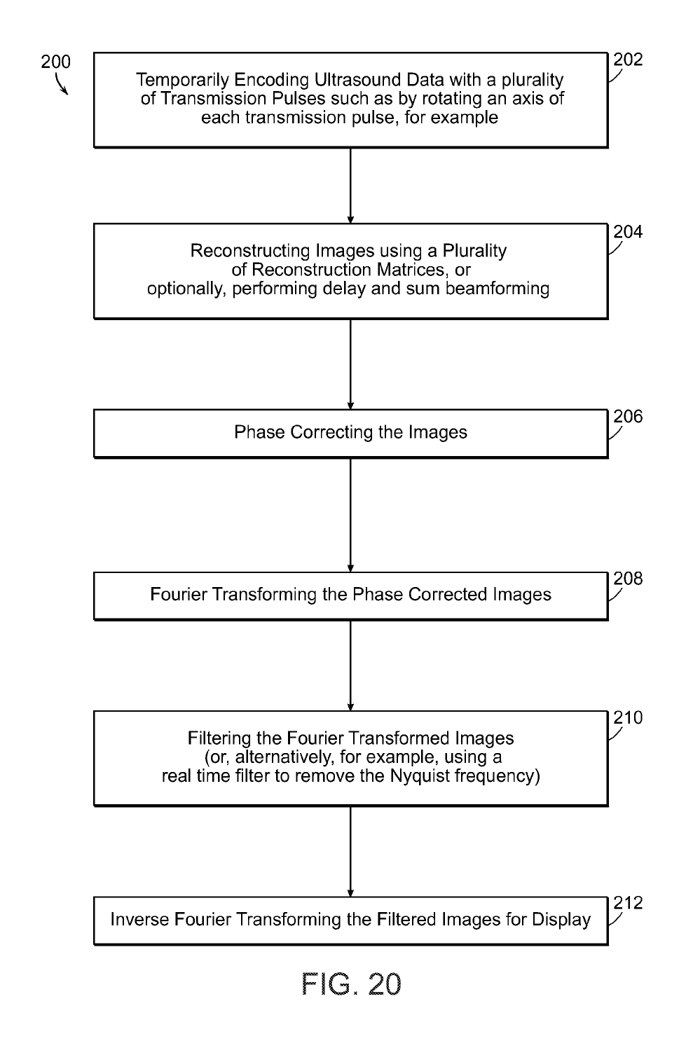

Illustrated in FIG. 20 is a method 200 of temporally encoding artifacts as described herein for ultrasound imaging. Transmission pulses are selected 202 that enable the removal of artifacts by a filtering operation. This method can utilize the reconstruction matrix processing methods 204 described herein, or alternatively, can also be used with conventional delay and sum beamforming methods. The images can be phase corrected 206, Fourier transformed 208, filtered 210 and inverse Fourier transformed for further processing and display. Alternatively, a real time filter can be used to remove the Nyquist signal thereby avoiding additional processing time involved in converting to the Fourier domain.

The present invention has been described in terms of one or more preferred embodiments, and it should be appreciated that many equivalents, alternatives, variations, and modifications, aside from those expressly stated, are possible and within the scope of the invention.

1. A method for producing an ultrasound image of an object using an ultrasound imaging system comprising:
acquiring RF ultrasound image signals with a transducer array;
digitizing the RF ultrasound image signals to form digitized RF ultrasound data; and
processing the digitized RF ultrasound data with a data processor, the processing step including forming an ultrasound image from the digitized RF ultrasound image data using a representation that includes a spatially varied regularization component.

2. The method of claim 1 further comprising generating at least one image of a region of interest in the object, the image having a plurality of pixels.

3. The method of claim 1 wherein the representation includes a reconstruction matrix and the processing step further comprises multiplying the reconstruction matrix with a data matrix that includes at least one ultrasound data point.

4. The method of claim 1 wherein the processing step further comprises:
using a numerical solver to process a first set of pixel data for a first image without generating a reconstruction matrix;
generating a second set of pixel data to form at least a second image using the first set of pixel data and the second set of pixel data.

5. The method of claim 1 further comprising acquiring second ultrasound image data using at least a second ultrasound transmission pulse that differs from a first ultrasound transmission pulse that generated a first ultrasound image.

6-8. (canceled)

9. The method of claim 8 further comprising varying the regularization component as a function of depth within the region of interest.

10. The method of claim 5 further comprising adjusting a phase component.

11. (canceled)

12. The method of claim 5 further comprising Fourier transforming and filtering image data.

13. The method of claim 1 further comprising performing time gain compensation on acquired RF ultrasound image data.

14. The method of claim 1 wherein the processing step comprises retrieving the spatially varied regularization component from a memory and computing the ultrasound image.

15. (canceled)

16. The method of claim 1 wherein the processing step further comprises using a wavepacket function defined by a plurality of pulse parameters including a field of view (FOV) and pulse voltage.

17. The method of claim 1 wherein the representation comprises a reconstruction matrix having unequal diagonal elements.

18. The method of claim 5 further comprising rotating a transmission pulse axis through a region of interest being scanned; and
phase compensating ultrasound data detected during axis rotation.

19-30. (canceled)

31. The method of claim 1 further comprising calculating a distribution of speed of sound within a region of interest.

32-34. (canceled)

35. A system for producing an ultrasound image of an object using an ultrasound imaging system comprising:
a transducer array for acquiring ultrasound signals;
an ultrasound system including a data processor that processes RF ultrasound image data, the data processor being operative to generate an ultrasound image computed from the RF ultrasound image data and a representation that includes a spatially varied regularization component; and
a display connected to the data processor that displays at least one ultrasound image having a plurality of pixels.

36. (canceled)

37. The system of claim 35 wherein the representation includes a reconstruction matrix and the processing step further comprises multiplying the reconstruction matrix with a data matrix that includes at least one ultrasound data point.

38. The system of claim 35 further comprising a numerical solver to process a first set of pixel data for at least one image without generating a reconstruction matrix.

39. The system of claim 35 further comprising a memory system that stores second ultrasound image data using at least a second ultrasound transmission pulse that differs from a first ultrasound transmission pulse that generated a first ultrasound image, the memory system being further operative to store a second set of pixel data for at least a second image.

40-41. (canceled)

42. The system of claim 35 wherein the transducer array comprises a linear or 2D transducer array for imaging a region of interest in the object, the transducer array being operative to emit a transducer pulse sequence for generating a plurality of images wherein the data processor adjusts a phase component of imaged data.

43. The system of claim 35 wherein the regularization component varies as a function of depth within the region of interest.

44. (canceled)

45. The system of claim 35 further comprising a transmitter for imaging using a plurality of focal depths within the object.

46. The system of claim 39 wherein the data processor is programmed with instructions for Fourier transforming and filtering image data.

47. The system of claim 35 further comprising a time gain compensation circuit to compensate RF ultrasound signals and an A/D converter to digitize the RF ultrasound data.

48. (canceled)

49. The system of claim 35 further comprising a memory that stores a wavepacket function defined by a plurality of pulse parameters including a field of view (FOV) and pulse voltage.

50. (canceled)

51. The system of claim 35 wherein the representation comprises a regularization matrix having unequal diagonal elements.

52-53. (canceled)

54. A system for ultrasound imaging comprising:
a transducer array for acquiring ultrasound signals over a time period in response to a plurality of varying transmission pulses that encode artifacts in detected ultrasound signals data;
an ultrasound system including on A/D converter that digitizes the detected ultrasound signals to form ultrasound data and a data processor that processes the ultrasound data with a filter to remove encoded artifacts from a plurality of ultrasound images.

55. The system of claim 54 further comprising a transmitter connected to the transducer array that is operative to rotate a transmission pulse axis through a region of interest being scanned by a transducer array.

56. The system of claim 55 wherein the data processor phase compensates ultrasound data detected during axis rotation.

57. The system of claim 54 further comprising a computer program stored in a memory system that applies a threshold to remove encoded artifacts, the memory system storing scan parameters such that the transducer array is actuated to scan a plurality of different focal locations within a region of interest.

58-60. (canceled)

61. The system of claim 54 further comprising a wavepacket function stored in a memory to generate the reconstruction matrix that is used to process ultrasound data.

62. (canceled)

63. The method of claim 1 wherein the representation comprises a plurality of patches such that the representation is sparse.

64. The system of claim 54 wherein the data processor is configured to generate an image without delay and sum beamforming.
